(19) United States
(12) Patent Application Publication
(10) Pub. No.: US 2026/0076983 A1
(43) Pub. Date: Mar. 19, 2026

(54) CANNABINOIDS COMPOSITIONS WITH POLYUNSATURATED FATTY ACID MONOGLYCERIDES AS A SPECIFIC COX-2 INHIBITOR, METHODS AND USES THEREOF

(71) Applicant: SCF PHARMA INC., SAINTE-LUCE (CA)

(72) Inventor: Samuel C. FORTIN, Sainte-Luce (CA)

(73) Assignee: SCF PHARMA INC., SAINTE-LUCE, QC (CA)

(21) Appl. No.: 19/107,867

(22) PCT Filed: Sep. 8, 2023
(86) PCT No.: PCT/CA2023/051189

§ 371 Date: Feb. 28, 2025

Related U.S. Application Data
(60) Provisional application No. 63/404,586, filed on Sep. 8, 2022.

(51) Int. Cl. A61K 31/00; A61K 31/232; A61P 29/00

(52) U.S. Cl. CPC A61K 31/658; A61K 31/232; A61P 29/00

(57) ABSTRACT

There are provided compositions comprising at least one compound chosen from compound of formula (I), compound of formula (II), compound of formula (III) and compound of formula (IV); and a cannabinoid for use as a COX-2 inhibitor.

### CROSS-REFERENCE TO RELATED APPLICATIONS

The present application claims the benefit of priority of co-pending U.S. Provisional Patent Application No. 63/404,586, which was filed Sep. 8, 2022, the content of which is incorporated herein by reference in its entirety.

### FIELD OF THE DISCLOSURE

The present document relates to the field of organic chemistry. More particularly, it relates to polyunsaturated fatty acid monoglyceride combinations with cannabinoids thereof. It also provides methods for inhibiting specifically the COX-2 enzyme without affecting the COX-1 enzyme.

### BACKGROUND OF THE DISCLOSURE

COX-2 is an inducible enzyme who has been used as a drug target for reducing pain and inflammation through is selective inhibition over COX-1. The first generation of marketed selective COX-2 inhibitor (rofocoxib and valdecoxib) were withdrawn from the market due to severe cardiac side-effect (S. X. Sun, K. Y. Lee, C. T. Bertram and J. L. Goldstein, Curr. Med. Res. Opin., 2007, 23, 1859-1866). Another specific COX-2 inhibitor, Celebrex, still available but with a side-effect boxed warning. Non-selective inhibitor such as ibuprofen, naproxen, nimesulide, diclofenac, and sunindac also cause severe side effects (I. L. Meek, M. A. Van de Laar and H. E. Vonkeman, Pharmaceuticals, 2010, 3, 2146-2162).

Finding a specific COX-2 inhibitor without the cardiac side effect would be a revolution in the field. The use of eicosapentaenoic acid, a cardioprotective fatty acid (N Engl J Med 2019; 380:11-22 DOI: 10.1056/NEJMoa1812792), and a cannabinoids all in one formulation made this possible.

### SUMMARY OF THE DISCLOSURE

There is provided a composition comprising at least one compound chosen from compound of formula (I), compound of formula (II), compound of formula (III) and compound of formula (IV):

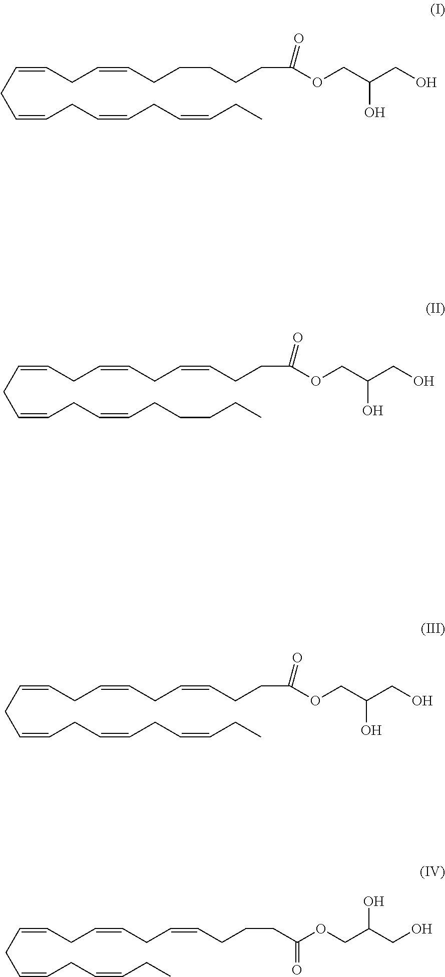 [SMILES: CC/C=C\C/C=C\C/C=C\C/C=C\C/C=C\CCCCCC(=O)OCC(O)CO ; CCCCC/C=C\C/C=C\C/C=C\C/C=C\C/C=C\CCC(=O)OCC(O)CO ; CC/C=C\C/C=C\C/C=C\C/C=C\C/C=C\C/C=C\CCC(=O)OCC(O)CO ; CC/C=C\C/C=C\C/C=C\C/C=C\C/C=C\CCCC(=O)OCC(O)CO]

and at least one cannabinoid for use as a COX-2 inhibitor.

There is also provided a composition comprising at least one compound chosen from compound of formula (I), compound of formula (II), compound of formula (III) and compound of formula (IV) and at least one cannabinoid chosen from Cannabidiol (CBD) and Cannabidiolic Acid (CBDA) and mixtures thereof for use as a COX-2 inhibitor.

There is also provided a composition comprising at least one compound chosen from compound of formula (I), compound of formula (II), compound of formula (III) and compound of formula (IV) and at least one of a cannabinoid and a cannabinoid extract for use as a COX-2 inhibitor.

There is also provided the use of a composition comprising at least one compound chosen from compound of formula (I), compound of formula (II), compound of formula (III) and compound of formula (IV):

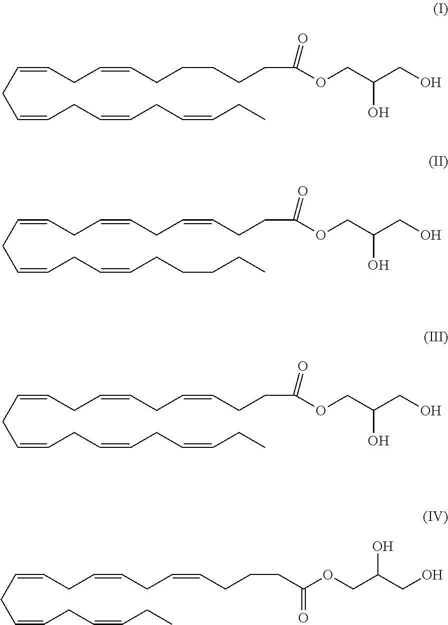 [SMILES: CC/C=C\C/C=C\C/C=C\C/C=C\C/C=C\CCCCCC(=O)OCC(O)CO ; CCCCC/C=C\C/C=C\C/C=C\C/C=C\C/C=C\CCC(=O)OCC(O)CO ; CC/C=C\C/C=C\C/C=C\C/C=C\C/C=C\C/C=C\CCC(=O)OCC(O)CO ; CC/C=C\C/C=C\C/C=C\C/C=C\C/C=C\CCCC(=O)OCC(O)CO]

and at least one cannabinoid, as a COX-2 inhibitor.

There is also provided the use of a composition comprising at least one compound chosen from compound of formula (I), compound of formula (II), compound of formula (III) and compound of formula (IV) and at least one cannabinoid chosen from Cannabidiol (CBD), Cannabidiolic Acid (CBDA) and mixtures thereof, as a COX-2 inhibitor.

There is also provided the use of a composition comprising at least one compound chosen from compound of formula (I), compound of formula (II), compound of formula (III) and compound of formula (IV) and at least one of a cannabinoid and a cannabinoid extract as a COX-2 inhibitor.

There is also provided the use of a composition comprising at least one compound chosen from compound of formula (I), compound of formula (II), compound of formula (III) and compound of formula (IV):

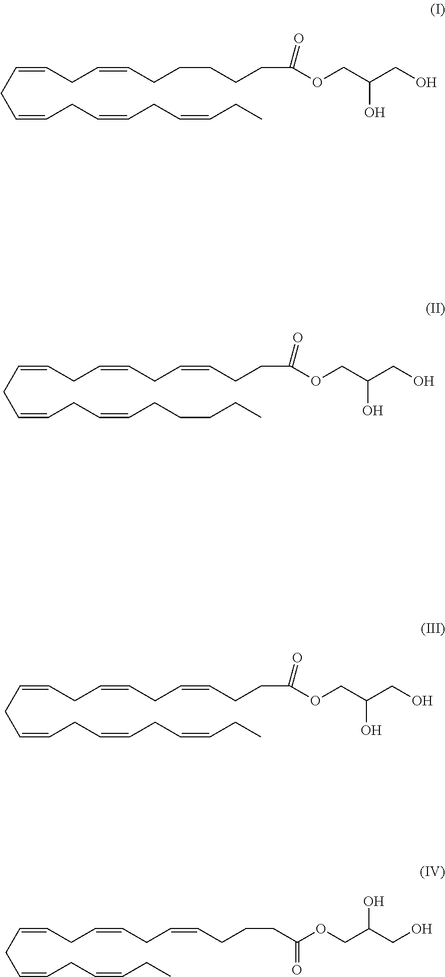 [SMILES: CC/C=C\C/C=C\C/C=C\C/C=C\C/C=C\CCCCCC(=O)OCC(O)CO ; CCCCC/C=C\C/C=C\C/C=C\C/C=C\C/C=C\CCC(=O)OCC(O)CO ; CC/C=C\C/C=C\C/C=C\C/C=C\C/C=C\C/C=C\CCC(=O)OCC(O)CO ; CC/C=C\C/C=C\C/C=C\C/C=C\C/C=C\CCCC(=O)OCC(O)CO]

and at least one cannabinoid, in the manufacture of a medicament for treating inflammation that is as a COX-2 inhibitor.

There is also provided the use of a composition comprising at least one compound chosen from compound of formula (I), compound of formula (II), compound of formula (III) and compound of formula (IV) and at least one cannabinoid chosen from Cannabidiol (CBD), Cannabidiolic Acid (CBDA) and mixtures thereof, in the manufacture of a medicament for treating inflammation that is as a COX-2 inhibitor.

There is also provided the use of a composition comprising at least one compound chosen from compound of formula (I), compound of formula (II), compound of formula (III) and compound of formula (IV) and at least one cannabinoid extract in the manufacture of a medicament for treating inflammation that is as a COX-2 inhibitor.

There is also provided a method for treating and/or preventing at least one disease chosen from encephalitis, myelitis, meningitis, arachnoiditis, neuritis, dacryoadenitis, scleritis, episcleritis, keratitis, retinitis, chorioretinitis, blepharitis, conjunctivitis, uveitis, labyrinthitis, mastoiditis, endocarditis, myocarditis, pericarditis, arteritis, phlebitis, capillaritis, asthma, sinusitis, rhinitis, pharyngitis, laryngitis, tracheitis, bronchitis, bronchiolitis, pneumonitis, pleuritis, mediastinitis, stomatitis, gingivitis, gingivostomatitis, glossitis, tonsillitis, sialadenitis/parotitis, cheilitis, pulpitis, gnathitis, esophagitis, gastritis, gastroenteritis, enteritis, colitis, enterocolitis, duodenitis, ileitis, caecitis, appendicitis, proctitis, hepatitis, ascending cholangitis, cholecystitis, pancreatitis, peritonitis, dermatitis, folliculitis, cellulitis, hidradenitis, arthritis, dermatomyositis, myositis, synovitis/tenosynovitis, bursitis, enthesitis, fasciitis, capsulitis, epicondylitis, tendinitis, panniculitis, osteochondritis: osteitis/osteomyelitis, spondylitis, periostitis, chondritis, nephritis, glomerulonephritis, pyelonephritis, ureteritis, cystitis, urethritis, oophoritis, salpingitis, endometritis, parametritis, cervicitis, vaginitis, vulvitis, mastitis, orchitis, epididymitis, prostatitis, seminal vesiculitis, balanitis, posthitis, balanoposthitis, chorioamnionitis, funisitis, omphalitis, insulitis, hypophysitis, thyroiditis, parathyroiditis, adrenalitis, lymphangitis, lymphadenitis, inflammaging and cancer. The method comprises administering an effective amount to a subject in need thereof of a COX-2 inhibitor composition that comprises at least one compound chosen from compound of formula (I), compound of formula (II), compound of formula (III) and compound of formula (IV);
and at least one cannabinoid.

There is also provided a method of use of a composition comprising at least one compound chosen from compound of formula (I), compound of formula (II), compound of formula (III) and compound of formula (IV) and at least one of a cannabinoid and a cannabinoid extract. The method comprises administering an effective amount to a subject in need thereof of the composition as a COX-2 inhibitor.

There is also provided a method for treating and/or preventing inflammation, the method comprises administering an effective amount to a subject in need thereof of a COX-2 inhibitor composition that comprises at least one compound chosen from compound of formula (I), compound of formula (II), compound of formula (III) and compound of formula (IV);
and at least one cannabinoid.

### BRIEF DESCRIPTION OF THE FIGURES

Further features and advantages will become more readily apparent from the following description of specific embodiments as illustrated by way of examples in the appended figures wherein:

FIG. 1 represents a comparative human cross-over case study COX-2 inhibition of two different compounds alone (compound of formula (IV) and CBD and a composition comprising CBD formulated in compound of formula (IV).

FIG. 2 represents a human case study of COX-1 and COX-2 inhibition of a composition comprising CBD formulated in compound of formula (IV).

FIG. 3 represents a comparative human cross-over case study COX-2 inhibition of two different compounds alone (compound of formula (IV) and a cannabinoids extract.

FIG. 4 represents a comparative human case study of COX-2 inhibition over 24 h of a composition comprising CBD formulated in compound of formula (IV).

### DETAILED DESCRIPTION OF THE DISCLOSURE

Further features and advantages of the previously-mentioned compounds will become more readily apparent from the following description of non-limiting

The term “cannabinoid” as used herein refers to at least one compound chosen from THC (Tetrahydrocannabinol), THCA (Tetrahydrocannabinolic acid), CBD (Cannabidiol), CBDA (Cannabidiolic Acid), CBN (Cannabinol), CBG (Cannabigerol), CBC (Cannabichromene), CBL (Cannabicyclol), CBV (Cannabivarin), THCV (Tetrahydrocannabivarin), CBDV, (Cannabidivarin), CBCV (Cannabichromevarin), CBGV (Cannabigerovarin), CBGM, (Cannabigerol Monomethyl Ether), CBE (Cannabielsoin), CBT (Cannabicitran) and mixtures thereof.

Further features and advantages of the previously-mentioned compounds will become more readily apparent from the following description of non-limiting examples.

For example, the compositions, uses and methods of the disclosure can be effective for treating and/or preventing at least one disease chosen from encephalitis, myelitis, meningitis, arachnoiditis, neuritis, dacryoadenitis, sderitis, episderitis, keratitis, retinitis, chorioretinitis, blepharitis, conjunctivitis, uveitis, labyrinthitis, mastoiditis, endocarditis, myocarditis, pericarditis, arteritis, phlebitis, capillaritis, asthma, sinusitis, rhinitis, pharyngitis, laryngitis, tracheitis, bronchitis, bronchiolitis, pneumonitis, pleuritis, mediastinitis, stomatitis, gingivitis, gingivostomatitis, glossitis, tonsillitis, sialadenitis/parotitis, cheilitis, pulpitis, gnathitis, esophagitis, gastritis, gastroenteritis, enteritis, colitis, enterocolitis, duodenitis, ileitis, caecitis, appendicitis, proctitis, hepatitis, ascending cholangitis, cholecystitis, pancreatitis, peritonitis, dermatitis, folliculitis, cellulitis, hidradenitis, arthritis, dermatomyositis, myositis, synovitis/tenosynovitis, bursitis, enthesitis, fasciitis, capsulitis, epicondylitis, tendinitis, panniculitis, osteochondritis: osteitis/osteomyelitis, spondylitis, periostitis, chondritis, nephritis, glomerulonephritis, pyelonephritis, ureteritis, cystitis, urethritis, oophoritis, salpingitis, endometritis, parametritis, cervicitis, vaginitis, vulvitis, mastitis, orchitis, epididymitis, prostatitis, seminal vesiculitis, balanitis, posthitis, balanoposthitis, chorioamnionitis, funisitis, omphalitis, insulitis, hypophysitis, thyroiditis, parathyroiditis, adrenalitis, lymphangitis, lymphadenitis, inflammaging and cancer.

For example, the composition can comprise the compound of formula (IV).

For example, the composition can comprise CBD.

For example, in any one of the compositions of the present disclosure, in any one of the uses of the present disclosure or in any of the methods of the present disclosure, the at least one compound can be a compound of formula (IV) compound and the at least one cannabinoid is CBD.

### Example 1

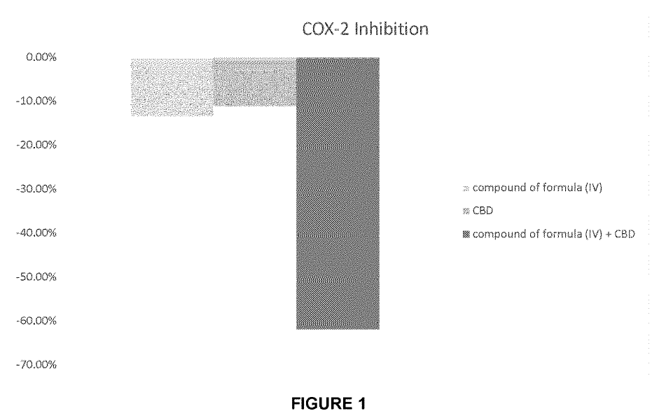

200 mg of cannabidiol (CBD) dissolved in 2.0 g of MCT oil, 2.0 g of compound of formula (IV) alone and 200 mg of cannabidiol (CBD) dissolved in 2.0 g of compound of formula (IV) were separately encapsulated in four (4) hard gel capsules (size 00) for a cross over COX-2 case study. A pilot cross-over case study was conducted in one volunteer over a three (3) week periods with one experiment per week. In the morning of the experiment, 10 ml of blood was collected in heparin tube from the volunteer fasted for 10 h. The four (4) hard gel capsules were swallowed with a glass of water and blood collection was repeated after 3.5 h. PGE₂ levels in lipopolysaccharide (LPS)-challenged human whole blood was measured as biochemical index for cyclooxygenase COX-2 activity (Brideau C, Kargman S, Liu S, Dallob A L, Ehrich E W, Rodger I W, Chan C C. A human whole blood assay for clinical evaluation of biochemical efficacy of cyclooxygenase inhibitors. Inflamm Res. 1996 February; 45(2):68-74. doi: 10.1007/BF02265118. PMID: 8907587). FIG. 1 shows the unexpected synergy between CBD and compound of formula (IV). CBD or compound of formula (IV) alone inhibit the COX-2 enzyme by 10% but the composition of cannabidiol (CBD) and compound of formula (IV) inhibit the enzyme by 60%, three times more than the combination of the two alone.

### Example 2

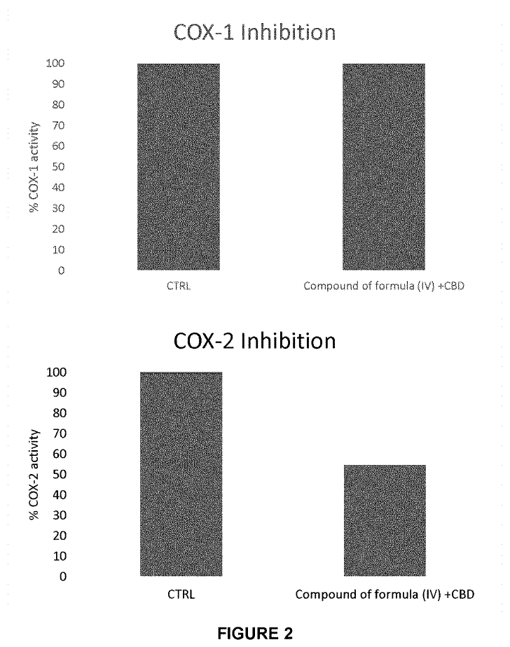

100 mg of cannabidiol (CBD) dissolved in 2.0 g of compound of formula (IV) was encapsulated in four (4) hard gel capsules (size 00) for COX-1 and COX-2 case study. A pilot case study was conducted in one volunteer. In the morning of the case study, 20 ml of blood was collected (1×10 ml serum tube and 1×10 ml heparin tube) from the volunteer fasted for 10 h. The four (4) hard gel capsules were swallowed with a glass of water and blood collection was repeated after 3.5 h. PGE₂ levels in lipopolysaccharide (LPS)-challenged human whole blood and TxB₂ levels following blood coagulation were measured as biochemical index for cyclooxygenase COX-2 and COX-1 activities (Brideau C, Kargman S, Liu S, Dallob A L, Ehrich E W, Rodger I W, Chan C C. A human whole blood assay for clinical evaluation of biochemical efficacy of cyclooxygenase inhibitors. Inflamm Res. 1996 February; 45(2):68-74. doi: 10.1007/BF02265118. PMID: 8907587). FIG. 2 shows the specificity of inhibition of the COX-2 enzyme over the COX-1 enzyme for the composition of cannabidiol (CBD) and compound of formula (IV).

### Example 3

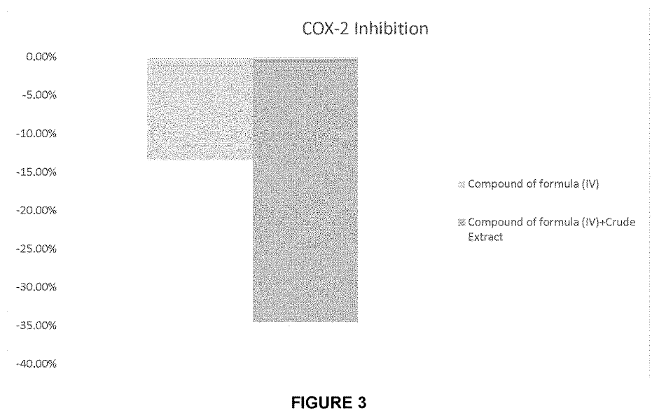

Cannabinoids extract containing 200 mg of cannabidiolic acid (CBA) dissolved in 2.0 g of compound of formula (IV) and 2.0 g of compound of formula (IV) were separately encapsulated in four (4) hard gel capsules (size 00) for a cross-over COX-2 case study. A pilot cross-over case study was conducted in one volunteer over a two (2) week periods with one experiment per week. In the morning of the experiment, 10 ml of blood was collected in heparin tube from the volunteer fasted for 10 h. The four (4) hard gel capsules were swallowed with a glass of water and blood collection was repeated after 3.5 h. PGE₂ levels in lipopolysaccharide (LPS)-challenged human whole blood was measured as biochemical index for cyclooxygenase COX-2 activity (Brideau C, Kargman S, Liu S, Dallob A L, Ehrich E W, Rodger I W, Chan C C. A human whole blood assay for clinical evaluation of biochemical efficacy of cyclooxygenase inhibitors. Inflamm Res. 1996 February; 45(2):68-74. doi: 10.1007/BF02265118. PMID: 8907587). FIG. 3 shows the mild inhibition of the COX-2 enzyme by compound of formula (IV) alone but the formulated cannabinoids extract in compound of formula (IV) inhibit the COX-2 enzyme more than twice the compound of formula (IV) alone.

### Example 4

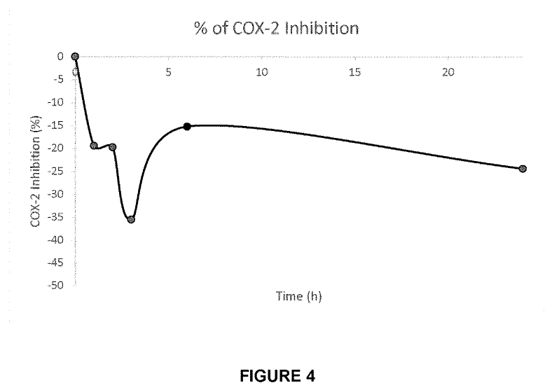

CBD (500 mg) was dissolved in 2.0 g of compound of formula (IV) and 2.5 g of compound of formula (IV) and was encapsulated in five (5) hard gel capsules (size 00) for a PK COX-2 inhibition case study. A pilot cross-over case study was conducted in one volunteer. In the morning of the experiment, 10 ml of blood was collected in heparin tube from the volunteer fasted for 10 h. The five (5) hard gel capsules were swallowed with a glass of water and blood collection was repeated after 1 h, 2 h, 3 h, 6 h, and 24 h. PGE₂ levels in lipopolysaccharide (LPS)-challenged human whole blood was measured as biochemical index for cyclooxygenase COX-2 activity (Brideau C, Kargman S, Liu S, Dallob A L, Ehrich E W, Rodger I W, Chan C C. A human whole blood assay for clinical evaluation of biochemical efficacy of cyclooxygenase inhibitors. Inflamm Res. 1996 February; 45(2):68-74. doi: 10.1007/BF02265118. PMID: 8907587). FIG. 4 shows that the formulated CBD in compound of formula (IV) inhibit the COX-2 enzyme over a 24 h period.

While the compounds, compositions, methods and uses thereof have been described in connection with specific embodiments thereof, it will be understood that they can be further modified and this application is intended to cover any variations, uses, or adaptations of the compounds, compositions, methods and uses thereof following, in general, the principles described in the present document and including such departures from the present disclosure as come within known or customary practice within the art to which the present document pertains and as may be applied to the features hereinbefore set forth, and as follows in the scope of the appended claims.

1. A composition comprising at least one compound chosen from compound of formula (I), compound of formula (II), compound of formula (III) and compound of formula (IV): 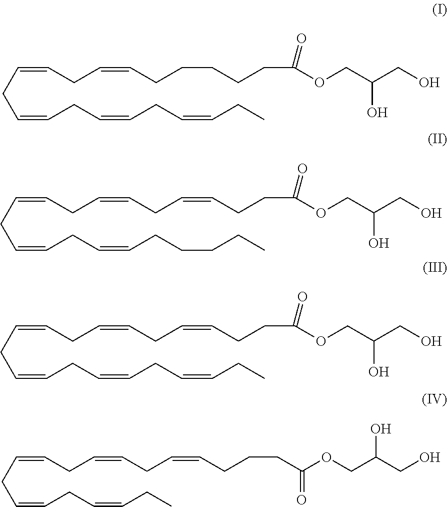 [SMILES: CC/C=C\C/C=C\C/C=C\C/C=C\C/C=C\CCCCCC(=O)OCC(O)CO ; CCCCC/C=C\C/C=C\C/C=C\C/C=C\C/C=C\CCC(=O)OCC(O)CO ; CC/C=C\C/C=C\C/C=C\C/C=C\C/C=C\C/C=C\CCC(=O)OCC(O)CO ; CC/C=C\C/C=C\C/C=C\C/C=C\C/C=C\CCCC(=O)OCC(O)CO]
and at least one cannabinoid for use as a COX-2 inhibitor.

2. The composition of claim 1, wherein the at least one cannabinoid is chosen from Cannabidiol (CBD), Cannabidiolic Acid (CBDA) and a mixture thereof, for use as a COX-2 inhibitor.

3-9. (canceled)

10. The composition of claim 1, wherein the composition comprises the compound of formula (IV).

11-13. (canceled)

14. A method for treating and/or preventing at least one disease chosen from encephalitis, myelitis, meningitis, arachnoiditis, neuritis, dacryoadenitis, scleritis, episcleritis, keratitis, retinitis, chorioretinitis, blepharitis, conjunctivitis, uveitis, labyrinthitis, mastoiditis, endocarditis, myocarditis, pericarditis, arteritis, phlebitis, capillaritis, asthma, sinusitis, rhinitis, pharyngitis, laryngitis, tracheitis, bronchitis, bronchiolitis, pneumonitis, pleuritis, mediastinitis, stomatitis, gingivitis, gingivostomatitis, glossitis, tonsillitis, sialadenitis/parotitis, cheilitis, pulpitis, gnathitis, esophagitis, gastritis, gastroenteritis, enteritis, colitis, enterocolitis, duodenitis, ileitis, caecitis, appendicitis, proctitis, hepatitis, ascending cholangitis, cholecystitis, pancreatitis, peritonitis, dermatitis, folliculitis, cellulitis, hidradenitis, arthritis, dermatomyositis, myositis, synovitis/tenosynovitis, bursitis, enthesitis, fasciitis, capsulitis, epicondylitis, tendinitis, panniculitis, osteochondritis: osteitis/osteomyelitis, spondylitis, periostitis, chondritis, nephritis, glomerulonephritis, pyelonephritis, ureteritis, cystitis, urethritis, oophoritis, salpingitis, endometritis, parametritis, cervicitis, vaginitis, vulvitis, mastitis, orchitis, epididymitis, prostatitis, seminal vesiculitis, balanitis, posthitis, balanoposthitis, chorioamnionitis, funisitis, omphalitis, insulitis, hypophysitis, thyroiditis, parathyroiditis, adrenalitis, lymphangitis, lymphadenitis, inflammaging and cancer,
said method comprising administering an effective amount to a subject in need thereof of a COX-2 inhibitor composition that comprises at least one compound chosen from compound of formula (I), compound of formula (II), compound of formula (III) and compound of formula (IV): 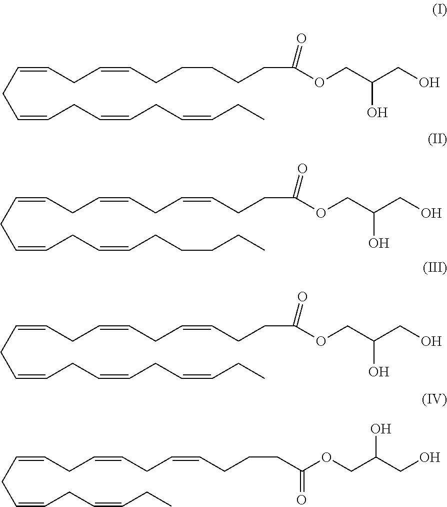 [SMILES: CC/C=C\C/C=C\C/C=C\C/C=C\C/C=C\CCCCCC(=O)OCC(O)CO ; CCCCC/C=C\C/C=C\C/C=C\C/C=C\C/C=C\CCC(=O)OCC(O)CO ; CC/C=C\C/C=C\C/C=C\C/C=C\C/C=C\C/C=C\CCC(=O)OCC(O)CO ; CC/C=C\C/C=C\C/C=C\C/C=C\C/C=C\CCCC(=O)OCC(O)CO]
and at least one cannabinoid.

15. (canceled)

16. A method for treating and/or preventing inflammation, said method comprising administering an effective amount to a subject in need thereof of a COX-2 inhibitor composition that comprises at least one compound chosen from compound of formula (I), compound of formula (II), compound of formula (III) and compound of formula (IV): 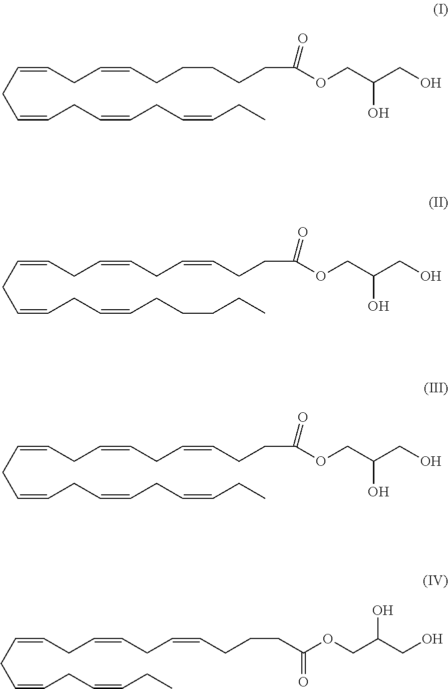 [SMILES: CC/C=C\C/C=C\C/C=C\C/C=C\C/C=C\CCCCCC(=O)OCC(O)CO ; CCCCC/C=C\C/C=C\C/C=C\C/C=C\C/C=C\CCC(=O)OCC(O)CO ; CC/C=C\C/C=C\C/C=C\C/C=C\C/C=C\C/C=C\CCC(=O)OCC(O)CO ; CC/C=C\C/C=C\C/C=C\C/C=C\C/C=C\CCCC(=O)OCC(O)CO]
and at least one cannabinoid.

17. The composition of claim 1, wherein the at least one compound is a compound of formula (IV) compound and the at least one cannabinoid is CBD.
